(19) United States
(12) Patent Application Publication
(10) Pub. No.: US 2006/0252917 A1
(43) Pub. Date: Nov. 9, 2006

(54) Methods for refolding conformationally constrained peptides

(75) Inventor: Grzegorz Bulaj, Salt Lake City, UT (US)

Correspondence Address: ROTHWELL, FIGG, ERNST & MANBECK, P.C., 1425 K STREET, N.W., SUITE 800, WASHINGTON, DC, 20005, US

(73) Assignee: University of Utah, Salt Lake City, UT (US)

(21) Appl. No.: 11/476,664

(22) Filed: Jun. 29, 2006

Related U.S. Application Data
(63) Continuation of application No. 10/926,348, filed on Aug. 26, 2004, now abandoned.
Application No. 10/926,348 is a continuation of application No. 10/067,988, filed on Feb. 8, 2002, now abandoned.
(60) Provisional application No. 60/267,192, filed on Feb. 8, 2001.

(51) Int. Cl. C07K 14/47

(52) U.S. Cl. 530/324; 530/408

(57) ABSTRACT

A method for refolding of conformationally constrained peptides is described, in which non-ionic detergents and other folding-additives increase yields of correctly folded bioactive peptides.

### CROSS-REFERENCE TO RELATED APPLICATION

The present application is a continuation of U.S. patent application Ser. No. 10/926,348 filed 26 Aug. 2004, which in turn is a continuation of U.S. patent application Ser. No. 10/067,988 filed 8 Feb. 2002. Ser. No. 10/067,988 is related to and claims priority under 35 USC §119(e) to U.S. provisional patent application Ser. No. 60/267,192 filed 8 Feb. 2001. Each application is incorporated herein by reference.

This invention was made with Government support under Grant No. PO1 GM48677 and Grant No. GM42494 awarded by the National Institute of General Medical Sciences, National Institutes of Health, Bethesda, Md. The United States Government has certain rights in the invention.

### BACKGROUND OF THE INVENTION

The present invention relates to a method for the production of short, disulfide-rich peptides. More specifically, the present invention relates to a method for refolding short, disulfide-rich peptides. The use of the present method allows for the production of biologically active peptides in significantly higher yields than prior art methods.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

Small, conformationally constrained peptides are ideal for a wide variety of biotechnology applications. Their small size facilitates access to specific target receptors. Specific cross-linking of disulfide bonds allows these small peptides to assume a relatively rigid structure that increases the probability of high affinity interaction with target molecules. Still, the variation of peptide structure afforded by variation in amino acid sequence in such peptides is enormous. Natural variants among peptides following this architectural design have been found to target a great diversity of target types. Molecules of this type have an expanding usefulness as agents capable of targeting a vast variety of receptors and ion channels on the surface of many different cell types. These molecules are useful in the design and testing of drugs targeting to variety of therapeutically important components, and in the design of agriculturally important agents. In addition, they have a more general potential as ligands used for interaction with broad classes of proteins and other biologically relevant macromolecules.

One group of small, conformationally constrained peptides are the conotoxins, which are small neuropeptides produced by mollusks of the genus Conus. Each of Conus species produce a large number (ranging from 50 to 200) of conotoxins which target a variety of ion channels and receptors, when injected with the venom into a prey (fish, worm or other snails). It is estimated that at least 50,000 unique conotoxins can be isolated from a total number of 500 different Conus species. Thus, conotoxins represent a very rich source of neuroactive peptides, many of them are studied as potential therapeutics for a number of neurological disorders. The most recent comprehensive reviews on their structure, function and potential therapeutic applications can be found in (McIntosh et al., 1999; Adams et al., 1999; Craig, 2001; Jones and Bulaj, 2000a; Jones and Bulaj, 2000b).

In addition to conotoxins, a number or small, disulfide-rich peptides with similar overall fold to that of conotoxins are found in different living species (reviewed by Norton and Pallaghy, 1998; Pallaghy et al., 1994). The examples include neurotoxins from spiders, but also proteinase inhibitors isolated from plants or fungi. Despite clearly diverse biological activities, these peptides have a common structural motif, so called the inhibitor cystine knot (Pallaghy et al., 1994), comprising three disulfide bonds and short backbone loops. Examples of such peptides include, but are not limited to Janus faced atrachatoxins (J-ACTXs) isolated from the Australian funnel web spider (Hadronyche versuta) (King et al., 2000), other spider toxins, such as agatoxins, grammotoxin, hanatoxins and huwentoxins (Grishin, 1999), scorpion toxins targeting sodium channels (Possani et al., 1999) and snake toxins, such as bungarotoxin and α-neurotoxins (Tsetlin, 1999). See, also Rappuoli and Montecucco (1997) for a comprehensive review of disulfide-rich toxins.

A large number of protein inhibitors of serine proteinases are also stablized by disulfide bonds and include such polypeptides as CMTI, EETI, Bowman-Birk inhibitors, ovomucoid third domains, serpins or Kunitz- and Kazal-type inhibitors (Bode and Huber, 1992). Other disulfide rich peptides have been isolated from plants, such as cyclic cystein know-type kalata B1, cycloviolacin O1, circulin A and varv peptide A (Craik et al., 2001).

These peptides exhibit a variety of biological activities, ranging from ion channel modulators and proteinase inhibitors to antiviral and antibacerial agents. Therefore, disulfide-rich, small peptides are a commercially very important group of compounds, and an efficiency of disulfide-coupled folding is often a determining factor in production costs and yields.

The small size of the peptides, i.e. those containing 10 to 30, or even more, amino acids, makes these peptides ideal for chemical synthesis. However, the presence of multiple cysteine residues increases the difficulty of obtaining biologically active peptides, i.e., peptides which have the same folding pattern as the native peptides. Obtaining small, conformationally constrained peptides which have the same folding pattern as the native peptides has presented a problem in the synthesis of these peptides. Obtaining such biologically active peptides is highly desirable for designing drug delivery systems which are targeted to specific protein receptors, or which interfere with the function of these receptor targets. These peptides have applications in biotechnology, particularly for the pharmaceutical industry and in agriculture. However, when such small peptides are synthesized by conventional synthetic methods, the formation of the disulfide bonds is non-specific and, when oxidized, the peptides appear to fold in many different disulfide configurations rather than in a specific biologically active form found in the naturally occurring peptides.

Linear peptides can be produced using two different strategies: (1) by chemical synthesis on solid support or in liquid phase or (2) by recombinant techniques with a variety of prokaryotic or eucaryotic hosts. There are many methods described in the literature that improve yields of production of short linear peptides and larger polypeptides, both by synthetic and recombinant methods (Chan and White, 2000; Sambrook et al., 1989; U.S. Pat. No. 5,231,011).

In order to produce a biologically active peptide from a linear peptide, specific pairs of cysteines must be oxidized, forming native disulfide bonds. This process is called disulfide-coupled folding (or refolding). To promote disulfide bond formation in peptides and proteins, a variety of oxidative reagents have been used. The most popular thiol-disulfide exchange reagents include glutathione (reduced GSH and oxidized GSSG), cystamine, cystine, 2-hydroxyethyl disulfide, β-mercaptoethanol. The other compounds used to oxidize peptide cysteines include oxygen (O₂) (so-called air oxidation), iodine, ferric ions (Fe³⁺) and many more.

For peptides that contain more than two cysteines, there are many possible combinations of disulfide arrangements. If non-native disulfides are formed in the final oxidation product, such misfolded peptides do not exhibit the native biological activity. Therefore, misfolding is one of the major problems that decrease folding yields, leading to low efficiency in producing bioactive peptides.

There are two basic strategies leading to the formation of native disulfide bonds: (1) direct oxidation and (2) stepwise regioselective deprotection of cysteine pairs. These strategies are reviewed in Annis et al. (1997) and Moroder et al. (1996). Both strategies have advantages and disadvantages in producing bioactive peptides, which are summarized in Table 1.

TABLE 1
Comparison Of Two Basic Strategies For
Oxidative Refolding Of Peptides

| Method | Advantages | Disadvantages |
|---|---|---|
| Direct | one-step reaction, | often observed low folding |
| oxidation | applicable to synthetic and | yields due to misfolding |
| | recombinant peptides | |
| Regioselective | selective formation of | multi-step reactions |
| deprotection | native disulfides | yields decrease as a number |
| | | of steps increase |
| | | only applicable to synthetic |
| | | peptides |

In order to improve folding yields, these two basic methods for producing bioactive, disulfide-rich, peptides have been further developed in many laboratories. Some innovations include: on-resin refolding (Altamirano et al., 1999), mini-chaperone assisted refolding (Ben-Zvi et al, 1998; published PCT application Nos. WO 98/13496, WO 98/24909, WO 99/02989 and WO 99/05163). In some instances, folding yields were improved by changing reaction conditions, such as concentration of oxidizing reagents, temperature or ionic strength.

In view of prior art difficulty in producing sufficiently high yields of conformationally constrained peptides which are biologically active, it is desirable to develop new methods for refolding peptides to produce biologically active peptides in significantly higher yield.

### SUMMARY OF THE INVENTION

The present invention relates to improving folding yields of small, disulfide rich peptides. In particular, the present invention relates to the use of so-called folding additives during the formation of disulfide bonds. More specifically, the present invention relates to a method comprising additive-assisted oxidative refolding of small conformationally constrained peptides. In one embodiment, an additive, such as a detergent, a water-soluble polymer, an osmolyte or other small molecule, is added to the folding reaction mixture. The method results in improved folding yields of the conformationally constrained peptides.

### BRIEF DESCRIPTION OF THE FIGURES

FIGS. 1A-1B show the structure of δ-conotoxins. FIG. 1A: Sequences and disulfide connectivity of four δ-conotoxins isolated from C. purpurascens (PVIA; SEQ ID NO:1), C. striatus (SVIE; SEQ ID NO:2), C. textile (TxVIA; SEQ ID NO:3) and C. gloriamaris (GmVIA; SEQ ID NO:4). Disulfide connectivity was determined only in PVIA and GmVIA (Shon et al., 1994; Shon et al., 1995). FIG. 1B: three dimension model structure of conotoxins belonging to the O-superfamily. This model illustrates the inhibitory cystine knot motif, comprising antiparallel triple-stranded β-sheet and three disulfide bonds that form four loop structure.

FIGS. 2A-2B show reversed-phase HPLC separations of PVIA. The separations were carried out using Vydac C18 analytical column in the gradient of acetonitrile as described herein. FIG. 2A: native PVIA isolated from venom of Conus purpurascence. FIG. 2B: folding mixture of PVIA in the absence of any folding enhancers. FIG. 2C: folding mixture of PVIA in the presence of 0.5% Tween 20.

FIG. 3 shows the results of screening various additives for improvement of folding yields for PVIA. The reactions were carried out at pH 8.7 and 0° C. for 24 hours in the presence of 1 mM cystamine and 1 mM GSH. Bars represent the yield (steady-state accumulation of the native PVIA, relative to the starting material, as determined by integration of HPLC peaks). The following are abbreviations used: DM—dodecyl maltoside; Tw60, Tw40, Tw20 are Tween 60, 40, 20, respectively; TrX100—Triton X100; PEG 8000—polyethylene glycol 8000; CD—cyclodextrins; PVA—polyvinyl alcohol; OG—octyl glucopyranoside, OTG—octyl thiolglucopyranoside; MeOH—methanol; EtOH—ethanol; IsopOH—isopropyl alcohol; ACN—acetonitrile.

FIG. 4 shows the effects of detergent type and concentrations on the folding yields of PVIA. Three different types of Tween detergents and dodecyl maltoside were tested. The reactions were carried out in the same way as described for FIG. 3.

FIG. 5 shows the kinetics of reductive unfolding of native PVIA with GSH as a function of detergent concentration. The reactions were performed in the presence of 10 μM PVIA, 0.5 mM GSH (reduced glutathione) at pH 8.7 and 0° C. After appropriate time, the aliquots were withdrawed and quenched with 5% formic acid. The concentration of the native PVIA was then determined by HPLC. The experimental points were fit into the exponential equation, yielding pseudo-first order rates.

FIG. 6 shows reversed-phase HPLC separations of folding mixtures for other δ-conotoxins: SVIE, TxVIA and GmVIA. The folding conditions were as described in detail herein. The retention time of the native peptide was determined independently in the coelution experiments, using the native peptides isolated from the venoms as a reference.

FIG. 7 shows reversed-phase HPLC separation of folding mixtures of MrVIB using a Vydac C18 analystical HPLC column. Elution of the peptides was carried out in the gradient of acetonitrile (in 0.1% trifluoracetic acid (TFA)) and monitored using an absorbance at 220 nm. FIG. 7A: linear reduced form of MrVIB. FIG. 7B: folding mixture in the absence of Tween detergent. The amount of native MrVIB is undetectable in this reaction. FIG. 7C: folding mixture with addition of Tween detergent after 24 hours. The peak corresponding to the native form comprised 37% of the total peak area. FIG. 7D: native, correctly folded MrVIB, which is biologically active. Note that the retention time of the native material is higher, as compared to that of the linear form, suggesting that the native peptide is more hydrophobic.

FIG. 8 shows a comparison of folding yields for folding of conotoxins from P-Superfamily: tx9A and gm9.1, after 12 hours of folding with reduced and oxidized glutathione (2 mM and 1 mM, respectively), at pH 8.7 and at 25° C. Bar graph represents the accumulation of the native, active material, as expressed in the integrated peak areas (arbitrary units). The first two bars (A—no Tween, B—0.5% Tween 40) described folding of Tx9A, a conotoxin isolated from C. textile and containing two γ-carboxyglutamate (Gla) residues. As shown in the figure, addition of Tween had only small effect on the accumulation of native Tx9A. Folding of gm9.1 was more efficient in the presence of 0.5% Tween 40. C—no Tween, D—0.5% Tween 40.

### DETAILED DESCRIPTION OF THE INVENTION

The present invention is described with reference to the conformationally constrained peptides of the class of conotoxins. However, it is to be understood to be equally applicable to all small conformationally constrained peptides, including but not limited to the contotoxin, neurotoxin and proteinase inhibitor peptides previously described. In addition, pairs of Cys residues in these peptides may optionally be replaced pairwise with isoteric lactam or ester-thioether replacements, such as Ser/(Glu or Asp), Lys/(Glu or Asp), Cys/(Glu or Asp) or Cys/Ala combinations. Sequential coupling by known methods (Hargittai et al., 2000; Barnay et al., 2000; Hruby et al., 1994; Bitan et al., 1997) allows replacement of native Cys bridges with lactam bridges. Synthetic methodologies to facilitate amide or ester bridge formation, which are well known to those in the art (e.g., HATU/NMM/DMF combination for amide bond formation), can thus be used to introduce such synthetic modification, while retaining cysteine protection. In addition thioester, retro-thioester, thioether or retro-thioethers may be used. Thioether analogs may be readily synthesized using halo-Ala residues commercially available from RSP Amino Acid Analogues. In addition, individual Cys residues may be replaced with homoCys, seleno-Cys or penicillamine, so that disulfide bridges may be formed between Cys-homoCys or Cys-penicillamine, or homoCys-penicllamine and the like.

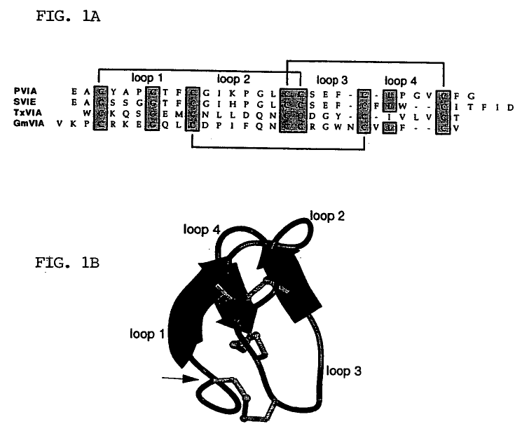

Based on the number and arrangement of cysteines, conotoxins are grouped into superfamilies and families. Examples of sequences and disulfide motifs are shown in Jones and Bulaj (2000b). The example of three-dimensional structure of conotoxins is shown in FIG. 1. It was shown in many cases that conotoxins belonging to the same family share identical disulfide arrangement and similar overall fold.

Conotoxins utilized as examples in this invention contain six cysteines, but belong to three different families: δ-, μO- and P-conotoxins. Two of them δ- and μO-conotoxins comprise a large number of sequences from O-superfamily. These peptides were shown to target sodium ion channels (Shon et al., 1994; Shon et al., 1995; Terlau et al., 1996; McIntosh et al., 1995). Their characteristic structural feature is a relatively large number of hydrophobic residues. On the other hand, conotoxins from P-superfamily are not particularly hydrophobic in nature (Lirazan et al., 2000). Their molecular target was suggested to be glycine receptors, based on their biological effects in mice.

In order to obtain biologically active conformationally constrained peptides it is necessary to form the proper native disulfide bonds. In the case 6-conotoxins, three native disulfide bonds must be formed (FIG. 1). The formation of disulfide bonds can be achieved by two different synthetic strategies: (1) a two-step oxidation with different orthogonal protection on the native pairs of cysteines or (2) a direct oxidative refolding. The two-step protocol has been previously applied to conotoxins belonging to the O-superfamily (Monje et al., 1993), as summarized in Table 2. However, in case of δ-conotoxins such as PVIA or GmVIA, the loss of material due to aggregation and precipitation at each folding step preclude efficient recovery of the native material. The one-step direct oxidation was successfully applied only to ω- and κ-conotoxins. Studies by Price-Carter et al., (1996a, 1996b) and Kubo et al., (1996) showed that disulfide-coupled folding for conotoxins, such as MVIIA or MVIIC could be optimized by changing reaction conditions: redox potential, temperature and ionic strength. Using standard two-step regioselective refolding strategy, the yields for δ- and μO-conotoxins were very low (not exceeding 5%) (Shon et al., 1994; McIntosh et al., 1995). In addition, early experiments showed that one-step direct oxidation methods produced very small quantities (TxVIA) or no/negligible amounts (MrVLA, MrVIB, PVIA, SVIE) of native conotoxins. Thus, an object of the present invention is to develop a method for refolding conformationally constrainted peptides which has improved refolding yields of such small, disulfide rich peptides.

TABLE 2
Summary of synthetic strategies for conotoxins
from the O-superfamily (C—C—CC—C—C).

| | | | ω- | |
|---|---|---|---|---|
| | δ-conotoxins | μO-conotoxins | conotoxins | κ-conotoxins |
| molecular | Na channels | Na channels | Ca channels | K channels |
| target | (slow down | (blockers) | (blockers) | (blockers) |
| | inactivation) | | | |
| chemical | non-polar | non-polar | positively- | positively- |
| feature | | | charged | charged |
| chemical | yes | yes | yes | yes |
| synthesis | | | | |
| two-step | yes | yes | yes | no attempt |
| oxidation | (poor yields) | (poor yields) | (high yields) | |
| direct | Yes | no success | yes | yes |
| oxidation | (detergent- | (unpublished) | (high yields) | (high yields) |
| | assisted | | | |
| | of the present | | | |
| | invention) | | | |

To improve refolding yields, a large number of folding additives, including detergents, water-soluble polymers, osmolytes, organic cosolvents and other small molecules were tested. Surprisingly, non-ionic detergents appeared to dramatically increase refolding yields for δ- and μO-conotoxins. In addition, it was found that temperature and organic cosolvents influenced refolding yield. For example, it was found that non-ionic detergents and low temperature were the most critical factors that influenced the folding yields of 6-contoxin PVIA. To date, non-ionic detergents have been used to increase refolding yields of larger proteins (Clark, 1998; Rudolph, 1996; Tandon and Horowitz, 1987), but have not been used for refolding short, disulfide rich peptides. Thus, the present invention relates to a detergent-assisted refolding of short, disulfide rich peptides. The studies described herein show that the mechanism of detergent-assisted folding for short, disulfide rich peptides is different from that of folding larger proteins: these results are described in Examples 3/4 and 6.

Effects of detergents on refolding of less hydrophobic conotoxins, such as GmVIA or those belonging to P-superfamily were also tested. Tween detergents were found to improve folding yields for those conotoxins as well (Examples 3/4 and 7). Therefore, the additive-assisted oxidative refolding of the present invention is applicable to other small, disulfide-rich peptides, exhibiting more polar character.

In accordance with the present invention, a method is provided for refolding small, disulfide-rich peptides. Small, disulfide-rich peptides is used herein to refer to peptides comprising 5 to 55 amino acids and containing two or more cysteines. Such peptides can be produced by chemical synthesis or by recombinant DNA techniques. The present invention comprises several embodiments with respect to the refolding mixture. In the first embodiment, the refolding reaction comprises a peptide in a solution containing redox reagents and a non-ionic detergent in an amount from about 0.001% to about 90%, preferably from about 0.01% to about 50%, more preferably from about 0.1% to about 10%. In a second embodiment, the refolding mixture comprises a peptide, redox reagents and a mixture of non-ionic detergents in the amount specified in the first embodiment. In a third embodiment the folding mixture comprises the peptide, redox reagents, non-ionic detergent(s) and a cosolvent (or a mixture of cosolvents) in an amount from about 0.1% to about 90%, preferably from about 1% to about 60%, more preferably from about 5% to about 40%.

The amount of redox reagents present in the folding mixture is in the range from about 0.01 mM to about 25 mM, preferably from about 0.1 mM to about 10 mM, more preferably from about 1 mM to about 5 mM. The oxidizing agent can be used alone, or in the mixture with the reducing agent. If a mixture of the reducing and oxidizing agents is used, the amount of reducing and oxidizing agents will be defined by their molar ratio ranging from 100:1 to 1:100, preferably from 20:1 to 1:10, more preferably from 10:1 to 1:2 (where the first and the second number refers to the reducing agent and the oxidizing agent, respectively).

The peptide is present in concentrations from about 0.1 μM to about 100 mM, preferably from about 1 μM to about 1 mM, more preferably from about 10 μM to about 100 μM. The peptide can be added to the refolding mixture directly or it can be immolized on a solid support. The peptide can be immobilized on a solid support subsequent to its synthesis and isolation or it can be immobilized on a solid support as a consequence of its synthesis. The peptide can be immobilized on the solid support covalently or non-covalently using techniques well known in the art.

Redox reagents which can be used are those which favor the formation of disulfide bonds or lactam or ether-thioester linkages. Examples of redox reagents include, but are not limited to, oxidized glutathione, reduced glutathione, cystine, cysteine, cystamine, β-mercaptoethanol and 2-hydroxyethyl disulfide. Examples of non-ionic detergents which can be used include, but are not limited to, polyoxyethylene and their derivatives (including polyoxylethylene sorbitans, ethers, esters), alkyl glucosides, maltosides and other alkyl derivatives of carbohydrates. Examples of cosolvents which can be used include, but are not limited to, common organic solvents, such as methanol, ethanol, isopropanol, acetonitrile, as well as other organic solvents comprising primary, secondary, tertiary, allylic, benzylic alcohols, ethers, aldehydes, ketones, carboxylic acids, amines, poly- and heterocyclic aromatic compounds, and a combination of these cosolvents.

Folding reactions specified in each embodiment may be conducted at a temperature in the range from about −10° C. to about 60° C., preferably in the range from about 0° C. to about 45° C., more preferably from about 0° C. to about 37° C. It is understood that duration of the folding reactions specified in each embodiment has to be determined and optimized individually for each peptide.

Folding reactions are preferably conducted in a buffered reaction mixture. Suitable buffers include, without limitation, tris, MOPS, MES, Tricine and glycine. The pH of the reaction mixture is from about 5.0 to about 12.0, preferably from about 6.0 to about 11.0, more preferably from about 7.0 to about 10.0. It is understood that the pH is determined and optimized individually for each peptide.

Without being bound by any specific theory or mechanism of action, the following discussion is based on the discoveries of the present invention. δ-Conotoxins create not only a protein folding challenge, but also a puzzle as a “reversed hydrophobic effect”. If the native form is more hydrophobic than the linear form or intermediates, what is the primary driving force that determines its proper folding? It is conceivable that the in vivo folding of 6-conotoxins is assisted by a specific cellular machinery, including chaperones and foldases, that has been evolved by Conus snails. However, a detail mechanism of this process still remains poorly understood (Price-Carter et al. 1996a,b; Kowalsman 2000). As disclosed herein, the major finding in this work was that the in vitro disulfide-coupled folding of a 6-conotoxin PVIA can be greatly improved in the presence of the non-ionic detergent and at low temperature.

There are several possible explanations (without being bound by any of them) of how detergents may affect the folding of this extremely hydrophobic conotoxin. For example, (1) by improving solubilization of the peptide in aqueous solutions, (2) by protecting an aggregation through partitioning between aqueous and micelles phases (3) by stabilization of the native form through direct interactions with the detergent molecule(s). The results of the present invention suggest that the detergent-assisted folding may involve direct interactions between the native δ-conotoxin and the detergent molecule. In addition, the solubilization of the peptide might also play an important role in increased folding yields of PVIA.

The interactions between Tween detergent and the native PVIA are very likely to have a hydrophobic character. The first evidence came from the dependence of the folding yield on the type of Tween detergents, which differ only by the length of hydrophobic hydrocarbon tail (FIG. 4). Tween 20, 40 and 60 are polyoxyethylene derivatives of sorbitan fatty acid esters having C12, C14 and C16 tails, respectively. The cmc values for the detergent are comparable: 5.9×10⁻⁵ M or 0.007% (Tween 20), 2.7×10⁻⁵ M or 0.004% (Tween 40) and 2.5×10⁻⁵ M or 0.003% (Tween 60). Thus, the observed correlation between the folding yields and the length of hydrocarbon group in the detergent is more likely to result from the chemical differences between the detergents, rather than from their tendency to form micelles. Indeed, for each detergent, the concentrations necessary for improving folding yields were found to be at least two orders of magnitude higher than the cmc values. Thus, the necessity of using such a large molar excess of the detergent over the peptide concentration suggested that the detergent-assisted folding could be driven by non-specific interactions between these two, most likely through the hydrocarbon part of the detergent molecules. In addition, low temperature greatly increased the efficiency of detergent-assisted folding, additionally supporting a suggestion that interactions between detergent and peptide may be of hydrophobic nature.

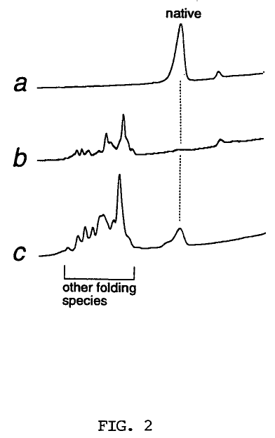

Non-polar interactions between the detergent and PVIA are not surprising, taking into account the hydrophobic nature of the native peptide. As mentioned earlier and illustrated in FIG. 2, the native PVIA is eluted at the highest acetonitrile concentrations, relative to the reduced form and the folded species. Thus, the formation of the native state is associated with a significant increase in the solvent-exposed non-polar surface area. Therefore, the mechanism of detergent-assisted folding of PVIA may involve stabilization of the native PVIA through the non-specific interactions between the hydrocarbon part of detergent and non-polar surface of the native PVIA. Indeed, the observed effects of Tween 40 on the reductive unfolding rates for PVIA suggest that this mechanism is plausible. In addition, folding of GmVIA which is less hydrophobic, also appeared to be the least influenced by Tween detergent. Taken together, our hypothesis is that the mechanism of detergent assisted folding involves stabilization of the native δ-conotoxins by hydrophobic interactions with the detergent.

The detergent-assisted folding was previously described for other polypeptides, such as rhodanese (Tandon and Horowitz, 1987), bacteriorhodopsin (Khorana, 1982), lactose transport protein (LacS) (Poolman, 1998) or human growth hormone (see also review by Zamadetta and Horowitz, 1994). In the comprehensive study of detergent-assisted refolding of rhodanese, Tandon and Horowitz (1987) observed that all types of detergents: non-ionic, ionic and zwitterionic were able to increase folding yields with different efficiency. These effects appeared to be strongly dependant on the detergent concentrations. For zwittergents, the maximum renaturation yields were found at concentrations between 2-5 times of cmc, but they dropped significantly when the detergents concentrations were above 5-10 fold cmc. Similar effects were observed for the ionic detergent CTAB. Even though the detergent concentrations had to exceed cmc for the maximum renaturation yields, the native rhodanese was not associated with micelles, suggesting that the mechanism of detergent assisted refolding involves weak interactions between detergent and rhodanese.

The mechanism of Tween-assisted refolding was also described in the case of human growth hormone rhGH (Kerwin 1998). In these studies, interactions between the protein and the Tween detergents were analyzed using spectroscopic and calorimetric studies. The detergent was shown to bind to the molten globule intermediate via hydrophobic interactions. In rhGH, molten globule is significantly more hydrophobic than the native state and more susceptible to aggregation. Thus, binding of detergent to the folding intermediate prevented aggregation, resulting in improved folding yields. In studies by Bam et al (1995, 1996, 1998), Tween was also shown to bind to the native rhGH with the stechiometry ranging between 2.5 to 4.1. The interactions between the protein and detergent had hydrophobic character, driven primarily by entropic rather than enthalpic effects. In this case, the binding of Tween to rhGH protected the protein against agitation-induced damage. Taken together, the interactions between molten globule or native hGH and Tween appear to have hydrophobic nature and are important for improving folding yields.

The hydrophobic interactions between non-ionic detergents and proteins are characterized by relatively low affinity (Sukow et al., 1980 and 1981). Similarly to the interactions between Tween and rhGH, binding of Triton X-100 to albumin showed only small enthalpy changes, indicating entropy-driven hydrophobic effects. Using equilibrium dialysis and titration calorimetry, Sukow et al., (1980, 1981) showed that Triton X-100 binds to serum albumin with millimolar affinity. The results of the present invention of the effects of the Tween series on the folding and unfolding of PVIA also suggested that the hydrophobic interactions between the detergent and the peptide might be in the millimolar affinity range. Increase in Tween concentration from 1% (8 mM) to 5% (38 mM) resulted in significant changes in the folding yields, as well as in the unfolding rates.

### EXAMPLES

The present invention is described by reference to the following Examples, which are offered by way of illustration and are not intended to limit the invention in any manner. Standard techniques well known in the art or the techniques specifically described below were utilized.

### Example 1

### Peptide Synthesis

Peptides were synthesized using standard Fmoc strategy. Cysteines were protected with either S-trityl or S-methoxytrityl groups. Other side chains were conventionally protected. The peptides were cleaved from the resin by treatment with reagent K (phenol: thionisole: TFA: water; ratio (1:3:93:1:2) for 3 hours at RT. The cleavage solution was filtered directly into the cold MTBE (−20° C.). The precipitated peptide was washed four times with cold MTBE and resuspended into solution by adding subsequently TFA, acetonitrile and water (final concentrations: peptide 1 mg/ml, 10% TFA, 60% ACN). For PVIA, the linear peptides was additionally incubated for 6-8 hours to remove the Boc protection group on tryptophane side chain. The linear form was purified on semi-preparative C18 column using reversed-phase HPLC. The initial conditions were the following: 20% of ACN in 0.1% TFA and linearly increased to 70% in 30 minutes. The elution of peptide was monitored by measuring the absorbance at 220 nm. The concentration of linear peptide was determined by measuring the absorbance at 274.5 nm (for Tyr containing peptides ε=1,420) or 279 nm (for Trp containing peptides, ε=5,600). The identity of synthetic material was confirmed by electrospray ionization mass spectrometry (ESI-MS) using Quatro II Micromass mass spectrometer and Masslynx software.

### Example 2

### Folding and Unfolding Reactions

Folding reactions were carried out in buffered solutions containing appropriate concentration of linear peptide, oxidative reagents and folding additives. The following buffer systems were used: MOPS-NaOH pH 7.0; Tris-HCl, pH 8.7; Glycine-NaOH, pH 10.0, each in 0.1 M concentration. In addition, each buffer contained 1 mM EDTA for inhibiting air oxidation (Wetlaufer 1980). Oxidative folding was carried out in the presence of reduced and oxidized glutathione or a mixture of cystamine and reduced glutathione. The folding reactions were initiated by injecting the linear peptide into the folding mixture comprising the buffer, EDTA, oxidative reagents and folding additive. The final peptide concentration was 10 μM. After appropriate time, an aliquot was withdrawn and the reaction was quenched by acidification using 10% formic acid (final concentration). Quenched reactions were analyzed by analytical reversed-phase separations on HPLC (Waters 900 system) using Vydac C18 column (4.6×250 mm) in the gradient of acetonitrile. The following separation conditions were used for all 6-conotoxins: linear gradient from initial 75% solvent A and 25% solvent B to final 25% solvent A and 75% of solvent B in 10 minutes, where solvent A=0.1% TFA, solvent B=90% ACN/0.1% TFA. Flow rate was 1 mL/min and the column temperature was 23° C. The elution was monitored by measuring the absorbance at 220 nm. Concentration of the native peptide was calculated from integration of HPLC peaks. The folding yields were calculated from the fraction of the native peptide relative to the other accumulated folding species. The reactions were at least duplicated and the accuracy of the folding yields was estimated to be within 2%. The total peak area was used to control whether precipitation occurs during folding. In order to control what peaks origin from folding additives, all buffer components and folding additives were separately injected on HPLC. Detergents, such as Brij or Trition X-100 were found to interfere with the HPLC peaks corresponding to the folding species. In these cases, the folding yields were calculated as a fraction of the native peptide, relative to the amount of the initial, reduced peptide.

Reductive unfolding kinetics was performed by adding reduced glutathione (0.5 or 1 mM) into the tube containing 0.1 M Tris-HCl, pH 8.7, 1 mM EDTA, 10 μM native PVIA. After appropriate time, the aliquots were withdrawn and treated with formic acid (10% final concentration). The concentration of the native PVIA was determined by HPLC. Experimental points were fit into a single exponential, yielding pseudo-first order rates (the reactions were carried out at 50 to 100 molar excess of the reducing reagent. In order to obtain the second-order rate constants, the pseudo-first order rates were divided by concentration of reduced glutathione.

### Example 3

### Analysis of Factors Influencing Folding of δ-Conotoxin PVIA

In order to examine factors that influence disulfide-coupled folding of δ-conotoxins, PVIA was used as a model peptide. PVIA is a 29 amino acids long peptide (FIG. 1A) that comprises only three charged residues (Glu1, Lys13 and Glu20) and as many as 13 non-polar side chains. Folding of PVIA is coupled to the formation of three native disulfide bonds: Cys3-Cys18, Cys10-Cys22 and Cys17-Cys27. Shon et al. (1994) reported chemical synthesis and oxidation of PVIA using a two-step protocol, in which the last step involved iodine oxidation of the acm-protected cysteines 10 and 22. The present invention is directed to an alternative method for producing native PVIA, the direct disulfide-coupled folding. In order to improve yields of the reaction, an extensive screening of folding-additives was carried out. In addition to folding enhancers, other factors that could influence folding, such as temperature, redox buffer or pH, were examined.

Folding reactions were performed in the similar way to that described by Price-Carter et al. (1996a, b). The purified reduced form of synthetic PVIA was prepared as described above, and the peptide concentration was determined by measuring the absorbance at 274.5 nm. To initiate the reaction, the reduced PVIA was transferred into a tube containing pH buffer (0.1 M Tris-HCl, pH 8.7 or 0.1 M MOPS-NaOH, pH 7.3), redox buffer (GSSG/GSG or cystamine/GSH) and appropriate folding additive or/and cosolvent. Final peptide concentration was 10 μM. After the folding reached equilibrium (24 hrs), the reaction was quenched by adding formic acid to 10% final concentration. The reaction mixture was separated on a reversed-phase column using HPLC.

FIG. 2 shows typical reversed-phase HPLC separations of PVIA folding mixtures on the analytical C18 Vydac column in the gradient of acetonitrile. The folding yields were estimated from the integration of HPLC peaks that corresponded to different folding species, including the native form. The direct oxidative folding resulted in approximately 1% yield, when the reaction was carried out in the presence of 1 mM oxidized and 2 mM reduced glutathione at pH 8.7 and 25° C. (these folding conditions were previously used by Price-Carter et al 1996 for the folding of ω-conotoxins). This remarkably low folding yield could be increased to approximately 3%, when the reaction was carried out in the presence of 1 mM cystamine (or 1 mM GSSG) and 1 mM GSH at 0° C. The synthetic folded form had the same retention time and a characteristic peak shape as that of the native peptide (FIG. 2). As mentioned earlier, the chromatographic behavior of the correctly folded PVIA was identical to that observed previously by Shon et al. (1994), i.e., the native form was eluted at much higher concentration of acetonitrile as compared to those of the linear form or folding intermediates.

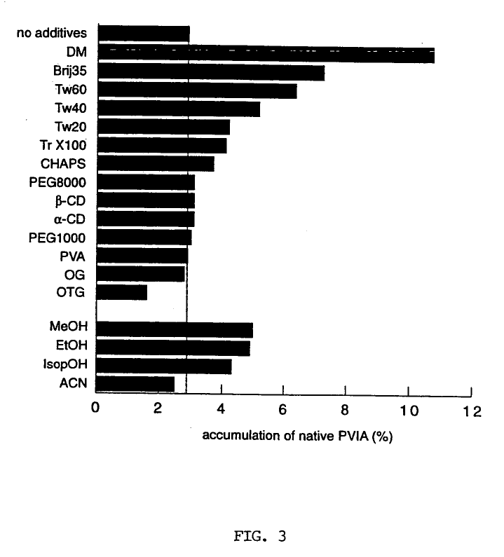

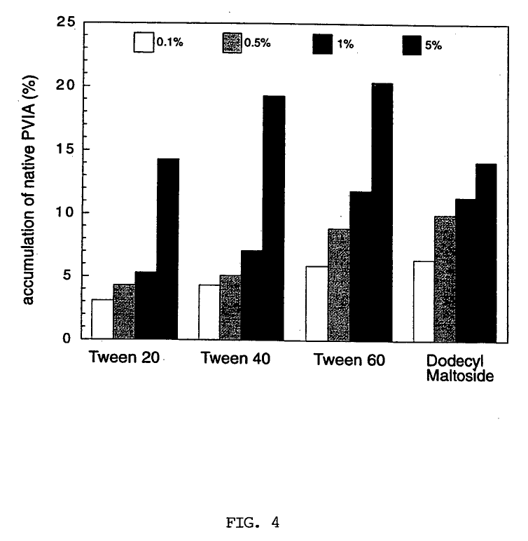

In order to screen for folding additives that could increase folding yields, a number of reactions were carried out in which the reduced PVIA was folded for 24 hours at 0 and 25° C. in the presence of 1 mM cystamine/1 mM GSH, pH 8.7 and 0.5% of different folding enhancers or 20% of organic cosolvents. FIG. 3 summarizes the effects of selected water-soluble polymers, detergents, other folding additives and organic alcohols on the folding yields. The non-ionic detergents, such as dodecyl maltoside, Brij 35 or Tween had significant effects on the accumulation of the native PVIA. The largest, 3-fold, increase in folding yields was observed in the presence of 0.5% dodecyl maltoside. Interestingly, other non-ionic detergents (octyl glucopyranoside and octyl thioglucopyranoside) did not facilitate PVIA folding. Similarly, water-soluble polymers (polyethylene glycol 8000 or 1000, polyvinyl alcohol) and cyclodextrins (α- and β-) were found without effects on folding yields, even though PEG 8000 appeared to improve folding of another 6-conotoxins TxVIA. Other additives: glycerol, sucrose or DMSO were without effect on folding yields.

This screening also identified organic cosolvents: methanol, ethanol and isopropanol that increased folding efficiency of PVIA (FIG. 3). At 20% concentration, all three cosolvents favored higher accumulation of the native PVIA. This effect was even larger at higher cosolvent concentrations. Methanol at 40% concentration was found to be the most effective cosolvent that increased folding yields up to 7%. Acetonitrile at concentrations 10-40% did not influence folding of PVIA. Interestingly, organic cosolvents had quite different effect on the detergent-assisted folding of PVIA. When folding was carried out in the presence of 5% Tween 40 at 0° C., addition of either methanol, ethanol, isopropanol or acetonitrile at concentrations above 20% decreased folding yields by more than two fold.

Temperature appeared to affect the influence of folding enhancers. Both, non-ionic detergents and organic cosolvents were more effective, when folding was carried out at 0° C. The effects of non-ionic detergents and temperature were not additive, suggesting that temperature played important role in the mechanism of detergent-assisted folding of PVIA.

Taken together, optimization of disulfide-coupled folding of PVIA identified several factors that significantly influenced accumulation of the correctly folded peptide. Selected non-ionic detergents, particularly Tween, organic cosolvents and low temperature were found to have the largest effects on the folding yields. Table 3 summarizes results of these experiments. The following recommended folding conditions for PVIA were established: 1 mM cystamine/1 mM GSH, 5% Tween 40 or 60, 10% methanol, pH 8.7, 0° C. for 24 hours. Under these conditions, folding yields were 23%.

TABLE 3
Optimization of Folding Conditions for δ-Conotoxin PVIA

| Factor^{a} | Type/Range Tested | Results/comments |
|---|---|---|
| Folding Additive | Water soluble polymers: PEG, PVA (0.5-5%) | Non-ionic detergents, in particular Tween series, |
| | Ionic and non-ionic detergents (0.1-10%) | at concentrations >1% significantly increase |
| | Other: cyclodextrins, dextrans, sucrose, glycerol, | folding yields |
| | DMSO | |
| Organic Cosolvents | MeOH, EtOH, isopropanol, acetonitrile (10-40%) | MeOH, EtOH, isopropanol at concentrations |
| | | 20%-40% increased folding yields; effect was |
| | | additive in the presence of non-ionic detergents. |
| Temperature | Range from 0° C. to 37° C. | Folding at 0° C. (e.g., in ice-bath) significantly |
| | | increased folding yield in presence or absence of |
| | | folding additives. |
| Redox Buffers | GSSG/GSH | Folding with 1 mM cystamine/1 mM GSH resulted |
| | Cystamine/GSH | in the highest yields of native peptide. |
| | (1 mM/1 mM; 1 mM/2 mM; 1 mM/5 mM) | |
| pH Buffer | 7.3 (0.1 M MOPS-NaOH) | No significant differences in the presence of |
| | 8.7 (0.1 M Tris-HCl) | folding additives. |
| | 10.0 (0.1 M Glycine-NaOH) | |
| Time | 0-72 hours | Equilibrium was achieved after 24 hours at 0° C. |
| | | or after 12 hours at 25° C. |

^{a}Tested at pH 8.7 and in presence of 1 mM cystamine/1 mM GSH (unless otherwise specified).

### Example 4

### Analysis of Detergent Effects

To further study a mechanism of the detergent-assisted folding, the effects of detergent type and concentrations on the folding yields of PVIA were examined. FIG. 4 shows the effects of a series of Tween detergents (20, 40, 60) and dodecyl maltoside on the PVIA folding yields at 0° C. These three types of Tween (polyoxyethylenesorbitan) had similar cmc values (critical micelle concentration), ranging from 0.003% to 0.007%, and differed only by the length of fatty acid ester: Tween 20 has the C₁₂ hydrocarbon tail (monolaureate), Tween 40 has C16 (monopalmitate) and Tween 60 has the C18 (monooleate) tail. As illustrated in FIG. 4, two correlations could be clearly distinguished: (1) detergent concentration dependence and (2) type of detergent dependence. The concentration dependence was similar for all three Tween detergents, with the highest yields observed at 5% concentrations. The most dramatic increase in the folding yields was observed for each Tween between 1% and 5% concentrations, where the values almost doubled. For dodecyl maltoside, the changes were not as dramatic within the tested range. Unfortunately, quantitative analysis of folding at detergent concentration above 5% was impossible, because higher detergents concentrations interfered with reversed-phase HPLC separations. Our preliminary experiments showed that the folding yields increased up to 30% in the presence of 10% Tween 20.

Another clear correlation was observed between type of the detergent and folding yields (FIG. 4). Modecyl maltoside appeared to be the most effective folding enhancer at lower detergent concentrations. However, at higher concentrations, Tween detergents were more efficient than dodecyl maltoside. Interestingly, when Tween 20, 40 and 60 detergents were compared, the smallest effects were systematically observed for Tween 20 and the largest for Tween 80. These results suggest that length of non-polar hydrocarbon tail in the detergent molecule plays an important role in facilitating formation of the native PVIA.

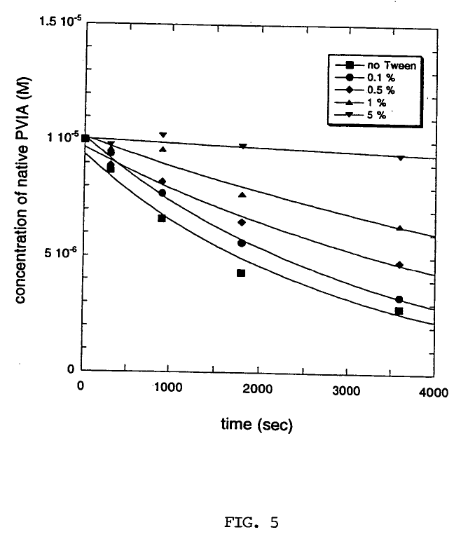

In order to test the influence of detergents on the stability of the native PVIA, we studied effects of Tween 40 on the reductive unfolding of PVIA in the presence of reduced glutathione. Kinetics of reductive unfolding has been often used as a determinant of the stability of cysteine-rich polypeptides (Mendoza et al, 1994; Price-Carter et al, 1996). Here, we tested the effects of increasing concentrations of Tween 40 on the reduction of PVIA by 0.5 mM at pH 8.7 and 0° C. FIG. 5 shows the time course of disappearance of the native PVIA (10 μM concentration) with 0.5 mM GSH at 0° C. Experimental points were analyzed using the non-linear exponential fit, yielding the pseudo-first order observed rate constants. The unfolding kinetics was clearly dependant on the detergent concentration: 0.021 s⁻¹ (no Tween), 0.019 s⁻¹ (0.1% Tween 40), 0.012 s⁻¹ (0.5%), 0.008 s⁻¹ (1%) and 0.001 s⁻¹ (5%). The effects of Tween 40 concentration on the reductive unfolding inversely correlated with their effects on the folding yields, suggesting that the higher accumulation of the native PVIA during folding could be accounted for by the stabilization of the correctly folded peptide by the detergent.

### Example 5

### Use of Refolding Method with Other Peptides

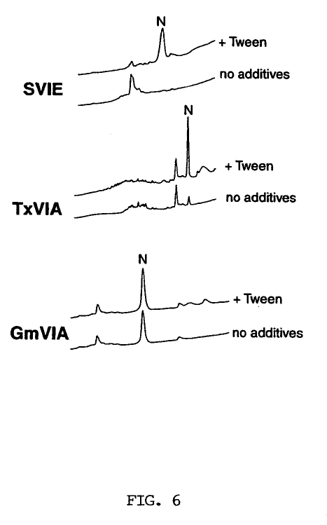

In addition to PVIA, the applicability of the detergent-assisted oxidative folding to other hydrophobic conotoxins: TxVIA, SVIE and GmVIA (see FIG. 1A) was tested. TxVIA and SVIE are similar to PVIA with respect to a number of charged and non-polar residues. On the other hand, GmVIA contains more positively charged residues. FIG. 6 shows HPLC separations of folding mixtures of all three conotoxins in the presence and absence of Tween 40 detergent. For TxVIA and SVIE, the detergent significantly increased the accumulation of the native peptide. Interestingly, lower temperature had only influence on the folding of TxVIA. In contrast to the detergent-assisted folding of TxVIA, SVIE or PVIA, the presence of Tween 40 had only small effects on the folding yields for GmVIA, suggesting that the amount of non-polar and charged residues may be important factor in determining the efficiency of the detergent-assistant folding.

### Example 6

### Method for the Production of MrVIB

This example illustrates the effect of Tween detergent on folding of conotoxin MrVIB. This conotoxin belongs to μO-family and O-superfamily, sharing the same disulfide arrangement with δ-conotoxins. However, the major differences include the primary sequence, as well as the size of the loops. The amino acid sequence of MrVIB is:

| | |
|---|---|
| ACSKKWEYCIVPILGFVYCCPGLICGFVCV | (SEQ ID NO:5) |

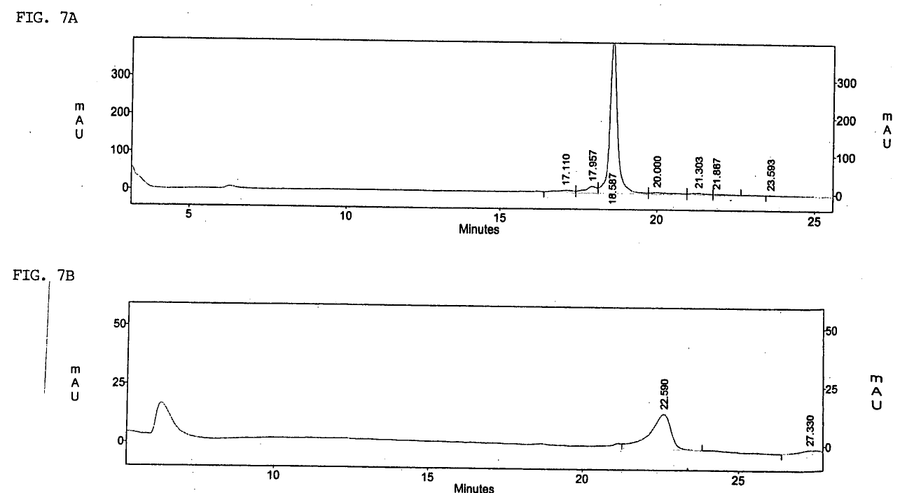

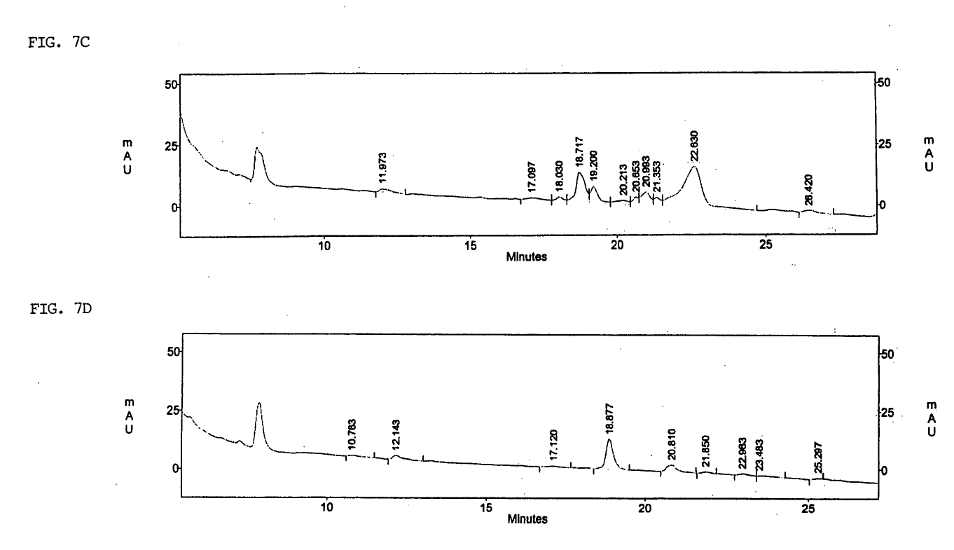

The folding experiments were essentially identical to those described in Example 3. Preparation of synthetic linear MrVIB included cleavage and purification of the peptide using reversed-phase HPLC. Standard folding mixtures included: buffer Tris-HCl pH 8.7, 0.1 mM EDTA, 1 mM GSH/2 mM GSSG, and 10 μM of linear MrVIB. In the absence of folding additives, this reaction did not produce any significant levels of native MrVIB. However, as illustrated in FIG. 7, addition of non-ionic detergent Tween 40 enhanced the accumulation of the native MrVIB.

### Example 7

### Method for the Production of Spasmodic P-Superfamily

This example describes detergent assisted folding of conotoxins belonging to the P-superfamily (Lirazan et al., 2000). These conotoxins contain six cysteines, but their cysteine pattern is different from that found in the O-superfamily. Sequences of these two peptides are shown below:

| | | |
|---|---|---|
| gm9.1 | SCNNSCQSHSDCASHCICTFRGCGAVN# | (SEQ ID NO:6) |
| tx9a | GCNNSCQγHSDCγSHCICTFRGCGAVN# | (SEQ ID NO:7) |

(where # describes amidated C-terminus and γ refers to γ -carboxyl glutamic acid).

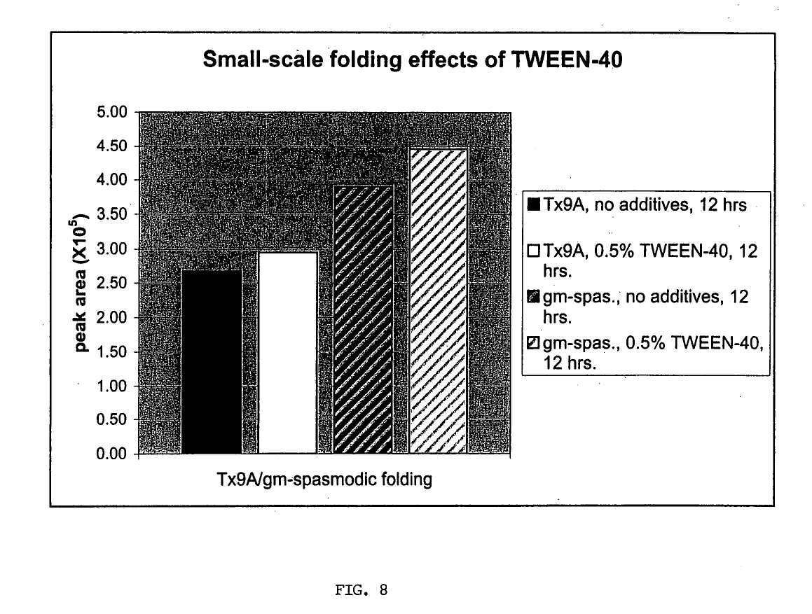

Synthetic peptides were prepared using standard Fmoc solid phase peptide synthesis. The linear forms were cleaved from the resin and purified using preparative reversed-phase HPLC separations. Folding reactions were performed using identical methods as described in Example 3. FIG. 8 shows a comparison of folding yields for a spasmodic peptide (C. gloriamaris) in the presence and absence of Tween detergent. It is clear from this graph that addition of detergent to the folding reaction increased refolding yields. In the case of conotoxins from C. textile, this effect was not obvious, even though these two sequences are very similar.

Examples 6 and 7 demonstrate that detergent-assisted folding can be applied to conotoxins from different families and to conotoxins having different cysteine patterns. Thus, the refolding method of the present invention can be used for folding other types of small, disulfide-rich peptides.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### BIBLIOGRAPHY

Adams, D. J. et al. (1999). Drug Develop. Res. 46:219-234.
Altamirano, M. M. et al. (1999). Nature Biotechnol. 17:187-191.
Annis, I. et al. (1997). Meth. Enzymology 289:198.
Bam N B et al. (1995) Pharm Res 12:2-11.
Bam N B et al. (1996) Biotech. Prog. 12:801-809.
Bam N B et al. (1998). J Pharm Sci. 87:1554-9.
Barnay, G. et al. (2000). J. Med. Chem.
Ben-Zvi, A. P. et al. (1998). Proc. Natl. Acad. Sci. USA 95:15275-15280.
Bitan, G. et al. (1997). J. Peptide Res. 49:421-426.
Bode, W. and Huber, R. (1992). Eur. J. Biochem. 204:433-451.
Chan, W. C. and White, P. D. (eds). (2000). Fmoc solid phase peptide synthesis: a practical approach, Oxford University Press, Oxford.
Clark, E. D. B. (1998). Cur. Opin. Biotechnol. 9:157-163.
Craig, A. J. (2000). J. Toxicol. 19:53-93.
Craik, D. J. et al. (2001). Toxicon—Toxin Reviews 39:43-60.
Grishin, E. (1999). Eur. J. Biochem. 264:276-280.
Hargittai, B. et al. (2000). J. Med. Chem. 43:4787-4792.
Hubry, V. et al. (1994). Reactive Polymers 22:231-241.
Jones, R. M. and Bulaj, G. (2000a). Cur. Opin. Drug Discovery Develop. 3:141-154.
Jones, R. M. and Bulaj, G. (2000b). Cur. Pharm. Design 6:1249-1285.
London, E and Khorana, H G (1982) J. Biol. Chem. 257, 7003-7011.
Kerwin, B. A., et al., (1998) J. Pharmaceutical Sci. 87: 1062-1068.
King et al. (2000) Nature Structural Biology 7: 505-513.
Kowalsman N. (2000). M. Sc. thesis, Feinberg Graduate School, Weizmann Institute: The Role of the Propeptide Domain in the Maturation of Conotoxins.
Kubo et al. (1996) Biopolymers 38:733-44.
Lirazan, M. B. et al. (2000). Biochemistry 39:1583-1588.
McIntosh, J. M. et al. (1995). J. Biol. Chem. 270:16796-16802.
McIntosh, J. M. et al. (1999). Meth. Enzymology 294:605-624.
Mendoza J. A. et al (1994) Biochemistry 33, 1143-48.
Monje, V D, et al. (1993) Neuropharmacology 32, 1141-1149.
Moroder, L. et al. (1996). Biopolymers 40:207-234.
Norton, R. S. and Pallaghy, P. K. (1998). Toxicon 36:573-583.
Pallaghy, P. K. et al. (1994). Protein Science 3:1833-1839.
Poolman, B et al. (1998) Biochemistry 37: 16410-15.
Possani, L. D. et al. (1999). Eur. J. Biochem. 264:287-300.
Price-Carter et al. (1996a) Biochemistry 35, 15537-15546.
Price-Carter et al. (1996b). Biochemistry 35, 15547-15557.
Rappuoli, R. and Montecucco, C. (eds) (1997). Guidebook to Protein Toxins and Their Use in Cell Biology, Oxford University Press, Oxford.
Rudolph, R. (1996). “Successful Protein Folding on an Industrial Scale.” In Protein Engineering: Principles and Practice, Cleland, J. L. and Craik, S. (eds), Wiley-Liss, Inc., pp. 283-298.
Sambrook, J. et al. (1989). Molecular Cloning: A Laboratory Manual, 2nd Ed., Cold Spring Harbor Laboratory, Cold Spring Harbor, N.Y.
Shon, K. J. et al. (1994). Biochemistry 33:11420-11425.
Shon, K. J. et al. (1995). Biochemistry 34:4913-4918.
Sukow W. W., et al. (1980) Biochemistry 19: 912-917.
Sukow W. W., et al. (1981) Physiol. Chem. & Physics 13: 455-459.
Tandon, S. and Horowitz P. (1987) J Biol Chem 262:4486-91.
Terlau, H. et al. (1996). Nature 381:33-38.
Tsetlin, V. (1999). Eur. J. Biochem. 264:281-286.
Wetlaufer et al, Protein Engineering 1: 141-146.
Zamadetta, G. and Horowitz, P. M. (1994). Anal. Biochem. 223:1-6.
U.S. Pat. No. 5,231,011.
PCT Published Patent Application No. WO 98/13496.
PCT Published Patent Application No. WO 98/24909.
PCT Published Patent Application No. WO 99/02989.
PCT Published Patent Application No. WO 99/05163.

What is claimed is:

1. A method for refolding a small, disulfide-rich peptide comprising:
(a) adding a peptide which comprises 5-55 amino acid residues containing two or more cysteines which form disulfide bonds to a refolding mixture which comprises
(i) a non-ionic detergent and
(ii) a redox reagent
(b) incubating the resulting mixture to form disulfide bonds in said peptide, whereby a refolded peptide is produced.

2. The method of claim 1, wherein said refolding mixture further comprises a cosolvent.

3. The method of claim 1, wherein the non-ionic detergent is selected from the group consisting of polyoxyethylenes, polyoxyethylene derivatives, alkyl derivatives of carbohydrates and mixtures thereof.

4. The method of claim 3, wherein said polyoxyethylene derviatives are selected from the group consisting of polyoxylethylene sorbitans, polyoxylethylene ethers and polyoxylethylene esters.

5. The method of claim 3, wherein said carbohydrate is selected from the group consisting of glucose and maltose.

6. The method of claim 2, wherein said cosolvent is selected from the group consisting of (i) methanol, (ii) ethanol, (iii) isopropanol, (iv) acetonitrile, (v) a solvent selected from the group consisting of primary, secondary, tertiary, allylic, benzylic alcohols, ethers, aldehydes, ketones, carboxylic acids, amines, poly- and heterocyclic aromatic compounds, and (vi) mixtures thereof.

7. The method of claim 1, wherein said redox reagent is selected from the group consisting of oxidized glutathione, reduced glutathione, cystine, cysteine, cystamine, α-mercaptoethanol and 2-hydroxyethyl disulfide.

8. The method of claim 1, wherein said redox reagent comprises an oxidizing agent and a reducing agent.

9. The method of claim 7, wherein said redox reagent comprises an oxidizing agent and a reducing agent.

10. The method of claim 1, wherein the refolding is performed at a temperature in the range of −10° C. to 60° C.

11. The method of claim 1, wherein the refolding is performed at a pH in the range of 5 to 12.

12. The method of claim 1, wherein the peptide is immobilized on a solid support.

13. A method for refolding a small, disulfide-rich peptide comprising:
(a) adding a peptide which comprises 5-55 amino acid residues containing two or more cysteines which form disulfide bonds at a concentration from about 0.1 μM to about 100 mM to a refolding mixture which comprises
(i) a non-ionic detergent in an amount from about 0.001% to about 90% and
(ii) a redox reagent in an amount from about 0.01 mM to about 25 mM
(b) incubating the resulting mixture to form disulfide bonds in said peptide, whereby a refolded peptide is produced.

14. The method of claim 13, wherein said refolding mixture further comprises a cosolvent in an amount from about 0.1% to about 90%.

15. The method of claim 13, wherein the non-ionic detergent is selected from the group consisting of polyoxyethylenes, polyoxyethylene derivatives, alkyl derivatives of carbohydrates and mixtures thereof.

16. The method of claim 15, wherein said polyoxyethylene derviatives are selected from the group consisting of polyoxylethylene sorbitans, polyoxylethylene ethers and polyoxylethylene esters.

17. The method of claim 15, wherein said carbohydrate is selected from the group consisting of glucose and maltose.

18. The method of claim 14, wherein said cosolvent is selected from the group consisting of (i) methanol, (ii) ethanol, (iii) isopropanol, (iv) acetonitrile, (v) a solvent selected from the group consisting of primary, secondary, tertiary, allylic, benzylic alcohols, ethers, aldehydes, ketones, carboxylic acids, amines, poly- and heterocyclic aromatic compounds, and (vi) mixtures thereof.

19. The method of claim 13, wherein said redox reagent is selected from the group consisting of oxidized glutathione, reduced glutathione, cystine, cysteine, cystamine, α-mercaptoethanol and 2-hydroxyethyl disulfide.

20. The method of claim 13, wherein said redox reagent comprises an oxidizing agent and a reducing agent.

21. The method of claim 20, wherein said redox reagent comprises an oxidizing agent and a reducing agent.

22. The method of claim 13, wherein the refolding is performed at a temperature in the range of −10° C. to 60° C.

23. The method of claim 13, wherein the refolding is performed at a pH in the range of 5 to 12.

24. The method of claim 13, wherein the peptide is immobilized on a solid support.

25. A method for preparing a small, disulfide rich peptide having a disulfide bridging pattern of a native peptide comprising:
(a) synthesizing a peptide which comprises 5-55 amino acid residues containing two or more cysteines which form disulfide bonds;
(b) isolating the synthesized peptide;
(c) adding the peptide to a refolding mixture which comprises
(i) a non-ionic detergent and
(ii) a redox reagent
(d) incubating the resulting mixture to form disulfide bonds in said peptide; and
(e) isolating the refolded peptide.

26. The method of claim 25, wherein the peptide is synthesized by chemical synthesis.

27. The method of claim 25, wherein the peptide is synthesized by a recombinant DNA technique.

28. The method of claim 25, wherein said refolding mixture further comprises a cosolvent.

29. The method of claim 25, wherein the non-ionic detergent is selected from the group consisting of polyoxyethylenes, polyoxyethylene derivatives, alkyl derivatives of carbohydrates and mixtures thereof.

30. The method of claim 29, wherein said polyoxyethylene derviatives are selected from the group consisting of polyoxylethylene sorbitans, polyoxylethylene ethers and polyoxylethylene esters.

31. The method of claim 29, wherein said carbohydrate is selected from the group consisting of glucose and maltose.

32. The method of claim 28, wherein said cosolvent is selected from the group consisting of (i) methanol, (ii) ethanol, (iii) isopropanol, (iv) acetonitrile, (v) a solvent selected from the group consisting of primary, secondary, tertiary, allylic, benzylic alcohols, ethers, aldehydes, ketones, carboxylic acids, amines, poly- and heterocyclic aromatic compounds, and (vi) mixtures thereof.

33. The method of claim 25, wherein said redox reagent is selected from the group consisting of oxidized glutathione, reduced glutathione, cystine, cysteine, cystamine, β-mercaptoethanol and 2-hydroxyethyl disulfide.

34. The method of claim 25, wherein said redox reagent comprises an oxidizing agent and a reducing agent.

35. The method of claim 33, wherein said redox reagent comprises an oxidizing agent and a reducing agent.

36. The method of claim 25, wherein the refolding is performed at a temperature in the range of −10° C. to 60° C.

37. The method of claim 25, wherein the refolding is performed at a pH in the range of 5 to 12.

38. The method of claim 25, wherein the peptide is immobilized on a solid support.

39. The method of claim 1, wherein a pair of cysteine residues is replaced pairwise with isoteric lactam or ester-thioether replacements

40. The method of claim 39, wherein siad replacement is selected from the group consisting of Ser/(Glu or Asp), Lys/(Glu or Asp), Cys/(Glu or Asp) and Cys/Ala combinations.

41. The method of claim 13, wherein a pair of cysteine residues is replaced pairwise with isoteric lactam or ester-thioether replacements

42. The method of claim 41, wherein siad replacement is selected from the group consisting of Ser/(Glu or Asp), Lys/(Glu or Asp), Cys/(Glu or Asp) and Cys/Ala combinations.

43. The method of claim 25, wherein a pair of cysteine residues is replaced pairwise with isoteric lactam or ester-thioether replacements

44. The method of claim 43, wherein siad replacement is selected from the group consisting of Ser/(Glu or Asp), Lys/(Glu or Asp), Cys/(Glu or Asp) and Cys/Ala combinations.


SEQUENCE LISTING (7 sequences)

SEQ ID NO: 1 - protein, 29 aa, Conus purpurascens
EACYAPGTFCGIKPGLCCSEFCLPGVCFG

SEQ ID NO: 2 - protein, 31 aa, Conus striatus
EACSSGGTFCGIHPGLCCSEFCFLWCITFID

SEQ ID NO: 3 - protein, 27 aa, Conus textile
WCKQSGEMCNLLDQNCCDGYCIVLVCT

SEQ ID NO: 4 - protein, 29 aa, Conus gloriamaris
VKPCRKEGQLCDPIFQNCCRGWNCVLFCV

SEQ ID NO: 5 - protein, 30 aa, Conus marmoreus
ACSKKWEYCIVPILGFVYCCPGLICGFVCV

SEQ ID NO: 6 - protein, 27 aa, Conus gloriamaris
SCNNSCQSHSDCASHCICTFRGCGAVN

SEQ ID NO: 7 - protein, 27 aa, Conus textile
  PEPTIDE (1)..(27): Xaa is gamma-carboxy-Glu
GCNNSCQXHSDCXSHCICTFRGCGAVN